SEQUENCE LISTING (14 sequences)

SEQ ID NO: 1 - DNA, 46 bp, Artificial Sequence
  mcheery F primer
gggcggatccggtggcagcggcggttcgatggtgagcaagggcgag

SEQ ID NO: 2 - DNA, 29 bp, Artificial Sequence
  mcheery R primer
ggtgctcgagcttgtacagctcgtccatg

SEQ ID NO: 3 - DNA, 31 bp, Artificial Sequence
  ZIF268F primer
tatacatatggaacgcccgtatgcttgccct

SEQ ID NO: 4 - DNA, 50 bp, Artificial Sequence
  ZIF268R primer
accggatccgcccgagccaccgctaccgccgtccttctgtcttaaatgga

SEQ ID NO: 5 - DNA, 37 bp, Artificial Sequence
  HMGF primer
aactacaaataccgaaaaggggagacaaaaaagaagt

SEQ ID NO: 6 - DNA, 22 bp, Artificial Sequence
  HMGR primer
tttagctcgatatgcagcaata

SEQ ID NO: 7 - DNA, 37 bp, Artificial Sequence
  forward primer for amplifying polynucleotide encoding eHU protein
gggggcggtggcggtaacaagactcaactgattgatg

SEQ ID NO: 8 - DNA, 21 bp, Artificial Sequence
  reverse primer for amplifying polynucleotide encoding eHU protein
cttaactgcgtctttcagtgc

SEQ ID NO: 9 - DNA, 29 bp, Artificial Sequence
  target polynucleotide immobilized on CM5 chip
gtgtgtgtgtgatctgcgtgggcggtaag

SEQ ID NO: 10 - DNA, 1056 bp, Artificial Sequence
  nucleotide sequence encoding probe binding specifically to the target sequence, “TGGGCG”
atggaacgcccgtatgcttgccctgtcgagtcctgcgatcgccgcttttctcgctcggatgagcttacccgccatatccgcatccacacaggccagaagcccttccagtgtcgaatctgcatgcgtaacttcagtcgtagtgaccaccttaccacccacatccgcacccacacaggcgagaagcccgggggcggtggcggtgtgccaacacctaagagacctcggggccgaccaaagggaagcaaaaacaagggtgctgccaagacccggaaaaccgggggtagcggtggctcgggcggatccggtggcagcggcggttcgatggtgagcaagggcgaggaggataacatggccatcatcaaggagttcatgcgcttcaaggtgcacatggagggctccgtgaacggccacgagttcgagatcgagggcgagggcgagggccgcccctacgagggcacccagaccgccaagctgaaggtgaccaagggtggccccctgcccttcgcctgggacatcctgtcccctcagttcatgtacggctccaaggcctacgtgaagcaccccgccgacatccccgactacttgaagctgtccttccccgagggcttcaagtgggagcgcgtgatgaacttcgaggacggcggcgtggtgaccgtgacccaggactcctccctgcaggacggcgagttcatctacaaggtgaagctgcgcggcaccaacttcccctccgacggccccgtaatgcagaagaagaccatgggctgggaggcctcctccgagcggatgtaccccgaggacggcgccctgaagggcgagatcaagcagaggctgaagctgaaggacggcggccactacgacgctgaggtcaagaccacctacaaggccaagaagcccgtgcagctgcccggcgcctacaacgtcaacatcaagttggacatcacctcccacaacgaggactacaccatcgtggaacagtacgaacgcgccgagggccgccactccaccggcggcatggacgagctgtacaagctcgagcaccaccaccaccaccactga

SEQ ID NO: 11 - protein, 15 aa, Artificial Sequence
  peptide linker
GGGGSGGGGSGGGGS

SEQ ID NO: 12 - protein, 6 aa, Artificial Sequence
  peptide linker
GGSGGS

SEQ ID NO: 13 - protein, 5 aa, Artificial Sequence
  peptide linker
GGGGG

SEQ ID NO: 14 - protein, 11 aa, Artificial Sequence
  peptide linker
GGGGSGGGGGS

(19) United States
(12) Patent Application Publication
(10) Pub. No.: US 2012/0164754 A1
(43) Pub. Date: Jun. 28, 2012

(54) KIT AND METHOD OF DETERMINING NUCLEOTIDE SEQUENCE OF TARGET NUCLEIC ACID

(75) Inventor: Joo-won RHEE, Yongin-si (KR)

(73) Assignee: SAMSUNG ELECTRONICS CO., LTD., Suwon-si (KR)

(21) Appl. No.: 13/302,585

(22) Filed: Nov. 22, 2011

(30) Foreign Application Priority Data
Dec. 22, 2010 (KR) 10-2010-0132821

(51) Int. Cl. G01N 33/53; B82Y 15/00

(52) U.S. Cl. 436/501; 977/774; 977/902

(57) ABSTRACT

A kit for determining a nucleotide sequence of a target nucleic acid, and a method of determining a nucleotide sequence of a target nucleic acid using the kit are disclosed.

### CROSS-REFERENCE TO RELATED APPLICATIONS

This application claims priority to Korean Patent Application No. 10-2010-0132821, filed on Dec. 22, 2010, and all the benefits accruing therefrom under 35 U.S.C §119, the disclosure of which is incorporated herein by reference.

### BACKGROUND

1. Field

The present disclosure relates to a kit including a target nucleic acid-binding protein, and a method of detecting a target nucleic acid by using the kit.

2. Description of the Related Art

Zinc finger proteins are a class of sequence-specific DNA binding proteins that include a polypeptide structural motif called a zinc finger domain (or motif) that may bind specifically to various target DNA sequences. Zinc finger domains may be used to construct various recombinant polypeptides that specifically recognize particular nucleotide sequences for detection. Zinc finger domains have a very strong binding force to DNA, and can be coupled to various fluorescence reporter proteins, which fluoresce at various wavelengths. Thus, research into specifically detecting target nucleic acids by using zinc finger proteins has been conducted.

Nucleic acid sequence determination has applications in single nucleotide polymorphism (SNP) discrimination, and pathogenic infection, viral infection, and genetic disease diagnosis. Existing nucleic acid diagnostic assays mostly involve amplification. Major drawbacks of amplification-based nucleic acid diagnostic assays are the likelihood of a false positive response caused by contaminants during the amplification and a highly probable error in predicting the concentration of the original unamplified target nucleic acid from amplification-based nucleic acid assay results.

Therefore, there is demand for probes that are specific to a target nucleic acid and so highly sensitive that they do not require amplification of the target nucleic acid for detection and diagnosis.

### SUMMARY

Provided herein are a kit for determining a nucleotide sequence of a target nucleic acid, and a method of determining the nucleotide sequence of a target nucleic acid by using the kit.

In an embodiment, the kit includes at least two nucleic acid-binding probes, wherein each nucleic acid-binding probe comprises a first protein that specifically binds a nucleotide sequence consisting of n bases, wherein n is an integer ranging from 3 to 12; a second protein, linked to a terminus of the first protein, that non-specifically binds to a minor groove of nucleic acid; and a detectable tag linked to a terminus of the second protein.

In an embodiment, the method includes contacting a target nucleic acid, whose nucleotide sequence is to be detected, with the at least two nucleic acid-binding probes of the kit; detecting a signal from the detectable tag linked to each of the at least two nucleic acid-binding probes; and converting the detected signal into a nucleotide sequence present in the target nucleic acid.

In an embodiment the method includes contacting a target nucleic acid with the at least two nucleic acid-binding probes of the kit; detecting a signal from the detectable tag linked to one of the nucleic acid-binding probes; and determining that the nucleotide sequence specifically bound by the nucleic acid-binding probe is present in the target nucleic acid when the signal from the detectable tag linked to the nucleic acid-binding probe indicates binding of the nucleic acid-binding probe to the target nucleic acid, or determining that the nucleotide sequence specifically bound by one of the nucleic acid-binding probes is absent from the target nucleic acid when the signal from the detectable tag linked to the nucleic acid-binding probe does not indicate binding of the nucleic acid-binding probe to the target nucleic acid.

Additional aspects of the invention will be set forth, in part, in the description which follows and, in part, will be apparent from the description, or may be learned by practice of the exemplary embodiments.

### BRIEF DESCRIPTION OF THE DRAWINGS

These and/or other aspects of the invention will become apparent and more readily appreciated from the following description of various exemplary embodiments, taken in conjunction with the accompanying drawings of which:

FIG. 1 is a schematic diagram of a polynucleotide encoding a nucleic acid-binding probe according to an embodiment of the invention showing the components of the encoded fusion protein and relative location of restriction sites used in constructing the polynucleotide;

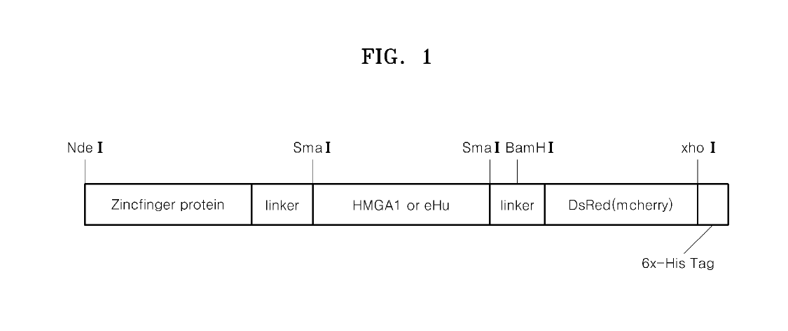

FIG. 2 is a schematic diagram of the expression vector pET12b-Zif268-HMGa1-cherry disclosed herein, which includes the polynucleotide encoding the nucleic acid-binding probe shown in FIG. 1, according to an embodiment of the invention, wherein the nucleotide sequence of the polynucleotide inserted into the expression vector is SEQ ID NO. 10; and

FIG. 3 is a sodium dodecyl sulfate-polyacrylamide gel electrophoresis (SDS-PAGE) image showing the fusion proteins expressed from the expression vectors pET12b-Zif268-HMGa1-cherry and pET12b-Zif268-eHU-cherry, according to an embodiment of the invention.

### DETAILED DESCRIPTION

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects of the present description.

Disclosed herein is a kit for determining a nucleotide sequence of a target nucleic acid, the kit including: at least two nucleic acid-binding probes, wherein each nucleic acid-binding probe comprises a first protein that specifically binds a nucleotide sequence consisting of n bases in the target nucleic acid; a second protein, linked to a terminus of the first protein, that non-specifically binds to a minor groove of nucleic acid; and a detectable tag linked to a terminus of the second protein. In an embodiment, when the first protein of the nucleic acid-binding probe is bound to its specific recognition sequence on the target nucleic acid, the second protein of the nucleic acid-binding probe can bind non-specifically to the minor groove of the target nucleic acid adjacent to the specific recognition sequence to which the first protein is bound.

In some embodiments, a nucleic acid-binding probe may include a first protein that specifically binds to a nucleotide sequence, the “specific recognition sequence”, consisting of n bases in the target nucleic acid, wherein n is an integer from 3 to 12. The specific recognition sequence of a nucleic-acid binding probe can be any arbitrary sequence selected from the 4ⁿ sequences possible for a nucleotide sequence of length n, wherein n is an integer from 3 to 12.

As used herein, the term “nucleic acid” refers to a polymer of nucleotides. The nucleic acid may include deoxyribonucleic acid (DNA; gDNA and cDNA) and/or ribonucleic acid (RNA), peptide nucleic acid (PNA), or locked nucleic acid (LNA). Nucleotides, which are the basic building blocks of nucleic acids, include not only natural nucleotides such as deoxyribonucleotide and ribonucleotide, but also artificial analogues including a modified sugar or base. Natural deoxyribonucleotides include one of the 4 types of bases: adenine (A), thymine (T), guanine (G), and cytosine (C). Ribonucleotides generally include a base which is C, A G, or uracil (U). The abbreviations, A, T or U, C, and G are used herein to describe either the base or the nucleotide in a nucleic acid sequence, according to context.

As used herein, the term “target nucleic acid” refers to a nucleic acid of interest whose nucleotide sequence is to be detected. Target nucleic acids may be genome DNA, mRNA, cDNA, and amplified DNA, but aspects of the present disclosure are not limited thereto.

The first protein may specifically bind to a specific recognition sequence consisting of n bases. The specific recognition sequence consisting of n bases may be selected from among the possible 4ⁿ n-mer nucleotide sequences, wherein the four bases may include adenine, guanine, cytosine, and thymine if the target nucleic acid is deoxyribonucleic acid (DNA), and in some embodiments, may include adenine, guanine, cytosine, and uracil, instead of thymine, if the target nucleic acid is ribonucleic acid (RNA). In some embodiments, n may be 6. If n is 6, the specific nucleotide recognition sequence may be selected from among the 4⁶ possible hexamer nucleotide sequences.

The first protein that specifically binds to the specific recognition sequence may include a nucleic acid-binding motif. In some embodiments the amino acid sequence of the first protein that specifically binds to the specific recognition sequence of the target nucleic acid may include at least one nucleic acid-binding motif selected from the group consisting of a zinc finger motif, a helix-turn-helix motif, a helix-loop-helix motif, a leucine zipper motif, the nucleic acid-binding motif of a restriction endonuclease, a TATA-binding protein (TBP) domain, and combinations thereof.

In some embodiments the amino acid sequence may include a zinc finger motif. The nucleic acid binding probe may include one to five zinc finger motifs, and in some embodiments, may include one to three zinc finger motifs, and in another embodiment, may include two zinc finger motifs.

The zinc finger motif may have any of the various zinc finger amino acid backbone structures known in the art, and in some embodiments, may be selected from the group consisting of a “Cys₂His₂” zinc finger, “Cys₄” zinc finger, “His₄” zinc finger, “His₃Cys” zinc finger, “Cys₃X” zinc finger, “His₃X” zinc finger, “Cys₂X₂” zinc finger, “His₂X₂” zinc finger (wherein X is a zinc ligating amino acid) and combinations thereof, which are non-limiting examples of zinc finger motif backbone structures.

The nucleic acid binding probe may specifically recognize and bind to a nucleotide sequence consisting of n bases in the target nucleic acid, wherein n may be an integer from 3 to 21, and in some embodiments, may be an integer from 6 to 18, and in some other embodiments, may be an integer from 6 to 9. In another embodiment, n may be 6. In some embodiments, a “Cys₂His₂” zinc finger motif may include an α-helical seven amino acid sequence that specifically recognizes a three nucleotide sequence. Zinc finger motifs may specifically recognize different nucleotide sequences. Nucleotide sequences specifically recognized by certain amino acid sequences of zinc finger motifs can be obtained using, for example, the internet-based program, Zinc Finger Tools (Mandell J G, Barbas CF 3rd. Zinc Finger Tools: custom DNA-binding domains for transcription factors and nucleases. Nucleic Acids Res. 2006 Jul. 1; 34 (Web Server issue):W516-23). For example, if n is 6, the first protein of the nucleic acid-binding probe may include two zinc finger motifs. The first protein may be constructed to be capable of specifically recognizing any of the possible 4⁶ nucleotide sequences, each including six bases, according to the combination of zinc finger motifs selected.

In some embodiments the zinc finger motif may be a wild-type zinc finger motif, a mutant type zinc finger motif, or a combination thereof. A mutant zinc finger motif may include about 1 to about 5 amino acid residues substituting for those of a wild-type zinc finger motif, and in some embodiments, may include about 2 to about 4 such substituted amino acid residues. These substituted amino acid residues may specifically bind to the nucleic acid.

A library of zinc finger motifs capable of specifically recognizing and binding to specific nucleotide sequences may be constructed by random mutation of an initial zinc finger motif on the gene level. For example, a phage display method by which a zinc finger motif library is displayed on a phage surface, a yeast one-hybrid method, a bacterial two-hybrid method, or a cell-free translation may be used to screen zinc finger motifs.

In one embodiment, the nucleic acid-binding probe may include a second protein that is linked to a terminus of the first protein and that can non-specifically bind to the minor groove, adjacent to the nucleotide sequence of the target nucleic acid bound by the first protein.

The second protein may bind to the minor groove in the specific recognition sequence of the first protein, consisting of n bases of the target nucleic acid, or to the minor groove within 20 bp from either terminus of the specific recognition sequence. Non-specific binding means that the second protein does not bind to a specific nucleotide sequence, but instead binds without sequence specificity. Examples of the second protein include a homeobox protein or homeobox domain, a high mobility group (HMG) protein or an HMG domain, a HU protein or HU class domain, a histone-fold protein or domain, a polymerase cleft protein or domain, and a protein including the non-sequence specific zincfinger xfin31, β-barrel CspA, an arginine-rich region, and a RGG motif.

The second protein may be linked to the N-terminus or the C-terminus of the first protein with or without a linker. The linker may be a non-peptide linker or a peptide linker. The linker will be described below.

In some embodiments the nucleic acid-binding probe may include a detectable tag, which is linked to a terminus of the second protein.

As used herein, the term “detectable label” refers to a moiety used to specifically detect a molecule or substance including the moiety from among the same type of molecules or substances without the moiety. The moiety can be an atom or a molecule. In some embodiments the detectable label may be a colored bead, an antigen determinant, an enzyme, a hybridizable nucleic acid, a chromophore, a fluorescent material, a phosphorescent material, an electrically detectable molecule, a molecule providing modified fluorescence-polarization or modified light-diffusion, a quantum dot, or the like. In addition, the detectable tag may be a radioactive isotope such as P³² or S³⁵, a chemiluminescent compound, a labeled binding protein, a heavy metal atom, a spectroscopic marker such as a dye, or a magnetic label. The dye may be a quinoline dye, a triarylmethane dye, phthalene, an azo dye, or a cyanine dye, but is not limited thereto. Suitable fluorescent materials may include Alexa Fluor 350, Alexa Fluor 430, Alexa Fluor 488, Alexa Fluor 532, Alexa Fluor 546, Alexa Fluor 568, Alexa Fluor 594, Alexa Fluor 633, Alexa Fluor 647, Alexa Fluor 660, Alexa Fluor 680, Cy2, Cy3.18, Cy3.5, Cy3, Cy5.18, Cy5.5, Cy5, Cy7, mCherry, Oregon Green, Oregon Green 488-X, Oregon Green, Oregon Green 488, Oregon Green 500, Oregon Green 514, SYTO 11, SYTO 12, SYTO 13, SYTO 14, SYTO 15, SYTO 16, SYTO 17, SYTO 18, SYTO 20, SYTO 21, SYTO 22, SYTO 23, SYTO 24, SYTO 25, SYTO 40, SYTO 41, SYTO 42, SYTO 43, SYTO 44, SYTO 45, SYTO 59, SYTO 60, SYTO 61, SYTO 62, SYTO 63, SYTO 64, SYTO 80, SYTO 81, SYTO 82, SYTO 83, SYTO 84, SYTO 85, SYTOX Blue, SYTOX Green, SYTOX Orange, SYBR Green YO-PRO-1, YO-PRO-3, YOYO-1, YOYO-3, and thiazole orange. In some embodiments the detectable tag may be included in the nucleic acid-binding probe to specifically detect binding to the specific nucleotide recognition sequence tof the first protein of the nucleic acid-binding probe.

In some embodiments the detectable tag may be linked to a terminus of the second protein via a linker. In some embodiments the linker may be attached to the N-terminus or the C-terminus of the specific sequence-binding first protein. The linker may be a non-peptide linker or a peptide linker.

The non-peptide linker may be any of various compounds that may be used as linkers in the art. A suitable linker may be selected based on the type of functional group in the protein (polypeptide) that binds to the target nucleic acid. In some embodiments the linker may be an alkyl linker or an amino linker. The alkyl linker may be a branched or non-branched, cyclic or acylic, substituted or unsubstituted, saturated or unsaturated, chiral, achiral or racemic mixture. In some embodiments the alkyl linker may have 2 to 18 carbon atoms. Other suitable alkyl linkers may include at least one functional group selected from among hydroxy, amino, thiol, thioether, ether, amide, thioamide, ester, urea, and thioether. The alkyl linker may be a 1-propanol linker, a 1,2-propandiol linker, a 1,2,3-propantriol linker, a 1,3-propandiol linker, a triethylene glycol hexaethylene glycol linker, a polyethylene glycol linker (for example, [—O—CH₂—CH₂—]ₙ, (n=1-9)), a methyl linker, an ethyl linker, a propyl linker, a butyl linker, or a hexyl linker.

The peptide linker may be any of various linkers that are widely used in the art, and in some embodiments, may be a linker including a plurality of amino acid residues. The peptide linker may keep the first protein and the second protein or the second protein and the detectable tag (for example, a fluorescent protein) apart from each other by a distance that is sufficient to allow the individual polypeptides to fold into appropriate secondary and tertiary structures. For example, the peptide linker may include Gly, Asn and Ser residues, and in some other embodiments, may include neutral amino acid residues, such as Thr and Ala. Amino acid sequences suitable for the peptide linker are known in the art. Suitable amino acid sequences may include (Gly₄-Ser)₃(SEQ ID NO: 11), (Gly₂-Ser)₂(SEQ ID NO: 12), Gly₅(SEQ ID NO: 13), and Gly₄-Ser-Gly₅-Ser (SEQ ID NO: 14). The linker may be unnecessary, and in some embodiments, may have various lengths, as long as it does not affect functions of the target sequence-binding protein and the detectable tag.

In some embodiments, the kit may include at least two nucleic acid-binding probes. The at least two nucleic acid-binding probes may each have an arbitrary specific recognition sequence of length n selected from the 4ⁿ possible n-mer sequences, wherein n is an integer selected from 3 to 12.

The kit may determine a nucleotide sequence of a target nucleic acid. The kit may also be used to detect the presence or absence of a nucleotide sequence in a target nucleic acid. The target nucleic acid, whose nucleotide sequence is to be detected, may have a length of from about 100 bp to about 10 Mb, and in some embodiments, may have a length of from about 1 kb to about 1 Mb. The target nucleic acid having a length within these ranges may include at least two nucleotide sequences, each consisting of n bases. Therefore, the kit may include at least two nucleic acid-binding probes, such that for each of the at least two n-mer nucleotide sequences of the target nucleic acid there is at least one nucleic acid binding probe present in the kit that specifically binds to that n-mer nucleotide sequence. The n-mer nucleotide sequences of the target nucleic acid may include any of the total possible 4ⁿ nucleotide sequences consisting of n bases. To detect all the possible n-mer nucleotide sequences that might be present in the target nucleic acid having a length within the above ranges, the kit may include a total of 4ⁿ nucleic acid-binding probes, at least one nucleic acid-bind probe to detect each of the possible 4ⁿ n-mer nucleotide sequences.

In some embodiments, the kit may include a reagent for stabilizing the nucleic acid-binding probes. For example, the kit may include a buffer solution known in the art. In some embodiments, the kit may be manufactured to have a plurality of separate packages or compartments.

Disclosed herein is a method of determining a nucleotide sequence of a target nucleic acid, the method including: contacting the target nucleic acid, whose nucleotide sequence is to be detected, with the at least two nucleic acid-binding probes in the kit; detecting a signal from the detectable tag linked to each of the at least two nucleic acid binding probes; and converting the detected signal into a nucleotide sequence. The detected signal from the detectable tag can be converted into a nucleotide sequence by identifying the nucleic acid-binding probe linked to the detectable tag and determining that the nucleotide sequence in the target nucleic acid is the specific recognition sequence of the identified nucleic acid-binding probe.

The method of determining the nucleotide sequence of the target nucleic acid, according to an embodiment, now will be described below.

The method may include contacting the target nucleic acid, whose nucleotide sequence is to be detected, with the at least two nucleic acid-binding probes in the kit.

In some embodiments, the contacting may be performed after a sample is acquired including the target nucleic acid. The sample may be a biological sample, or a non-biological sample. The biological sample may be any sample containing the target nucleic acid. Non-limiting examples of the biological sample include blood, tear drops, saliva, viruses, and microorganisms. The non-biological sample may be, for example, a DNA fragment amplified through nucleic acid amplification.

In some embodiments the contacting may be achieved by mixing the nucleic acid-binding probes and the sample itself or the target nucleic acid extracted from the biological sample in a liquid medium. The liquid medium may be any buffer solution known in the art, which is capable of maintaining the stability of the nucleic acid-binding probes and the target nucleic acid and of permitting binding between the nucleic acid-binding probes and their respective specific recognition sequences. The contacting allows the nucleic acid-binding motif of the first protein in the nucleic acid-binding probe to approach the target nucleic acid and allows the nucleic acid binding probe to specifically bind to the specific recognition sequence of the first protein in the nucleic acid-binding probe. The contacting may be followed by washing away any nucleic acid-binding probe that remains unbound. Through the above-described steps specific kinds of nucleic acid-binding probes, depending on the specific nucleotide recognition sequence of a given nucleic acid-binding probe, may be specifically bound to the nucleotide sequence of the target nucleic acid.

The target nucleic acid may be a double-stranded polynucleotide. Target nucleic acid having various lengths may be prepared by using methods known in the art. For example, the target nucleic acid may have a length of about 100 bp to about 10 Mb, and in some embodiments, may have a length of about 1 kb to about 1 Mb.

In some embodiments the target nucleic acid may further include a detectable tag. The target nucleic acid may be labeled with the detectable tag when being prepared. Suitable detectable tags are the same as those described above.

In some embodiments the method may include detecting a signal from the detectable tag linked to the nucleic acid-binding probes.

The signal generated from the detectable tag may be detected by using a detector. Depending on the nucleotide sequence of the target nucleic acid, different kinds of nucleic acid-binding probes may be bound. The different kinds of nucleic acid-binding probes may be labeled with different detectable tags. By distinguishing the signals detected from the different detectable tags, nucleotide sequences at different sites of the target nucleic acid may be identified.

In some embodiments, examples of signals generated from the detectable tag include a magnetic signal, an electric signal, a light-emitting signal such as a fluorescent or Raman signal, a diffusion signal, and a radioactive signal. Examples of the detection signal are the same as described above in conjunction with the detectable tag.

In some embodiments, detecting a signal from a detectable tag linked to at least two nucleic acid-binding probes may be performed when a biological sample including a target nucleic acid is contacted to the nucleic acid-binding probes. If the target nucleic acid is an isolated polynucleotide, the isolated polynucleotide may also be labeled with a detectable tag to detect a signal based on fluorescent resonance energy transfer (FRET) between the detectable tag bound to the target nucleic acid and a detectable tag bound to a nucleic acid-binding probe, thereby permitting determination of target nucleic acid bound to the nucleic acid-binding probe. “Isolated,” when used to describe the various polypeptides, nucleic acid-binding probe fusion proteins, or polynucleotides disclosed herein, means a polypeptide, fusion protein, or polynucleotide that has been identified and separated and/or recovered from a component of its natural environment. The term also embraces recombinant polynucleotides and polypeptides and chemically synthesized polynucleotides and polypeptides.

In some embodiments the method may further include converting the detected signal into the nucleotide sequence of the target nucleic acid. In some embodiments, the converting of the detected signal may be followed by outputting the nucleotide sequence converted from the detected signal to a user.

The nucleotide sequence of the target nucleic acid may be inferred from the signals generated from the detectable tags attached to the different kinds of nucleic acid-binding probes, which are bound to the target nucleic acid sequence. For example, if a nucleic acid-binding probe capable of specifically binding to a sequence of AAAAAA is labeled with a red fluorescent tag, and a nucleic acid-binding probe capable of specifically binding to a sequence of AAAAAT is labeled with a blue fluorescent tag, signals may be detected from the sites of the target nucleic acid being emitted in red and blue, and converted to nucleotide sequences (inferred to have those nucleotide sequences). If we use 4⁶ different nucleic acid binding probes, the nucleic acid binding probes can cover the entire nucleotide sequence of the target nucleic acid. Then, individual 6-mer nucleotide sequences which are read from some of the 4⁶ different nucleic acid binding probes can be ordered to provide the complete ordered nucleotide sequence of the target nucleic acid. The determined entire nucleotide sequence may be output to a user. Any suitable available detector and printer in the art may be used.

The present disclosure will be described in further detail with reference to the following examples. These examples are for illustrative purposes only and are not intended to limit the scope of the disclosure.

### Example 1

### Preparation of a Fusion Protein Including a Target Nucleotide Sequence-Binding Protein

In the present example a vector for expressing a fusion protein is prepared. The fusion protein includes a target nucleotide sequence-binding protein (zinc-finger protein), a protein (HMGa1 domain or eHU protein) that non-specifically binds to the minor groove of a nucleic acid, and a detectable tag (mCherry fluorescent protein, “mCherry”). The fusion protein is expressed using the vector and purified.

In order to synthesize the target nucleotide sequence-binding protein, a polynucleotide fragment coding for part of a (Gly₂Ser)₅ linker and a fluorescent protein (mCherry) was obtained by polymerase chain reaction (PCR) amplification. Amplification of the polynucleotide fragment was performed using pmCherry (Clontech, cat. no. 632522) as template, a mCherry F primer (SEQ ID NO. 1) coding for part of the (Gly₂Ser)₅ linker and also including a nucleotide sequence cleavable by BamHI, and a mCherry R primer (SEQ ID NO. 2) including a nucleotide sequence cleavable by XhoI. The amplification was performed using a GENEAMP® PCR System 9700 (Applied Biosystems) under the following PCR conditions: at 95° C. for 5 minutes; at 95° C. for 20 seconds; repeated 30 times at 68° C. for 2 minutes; at 68° C. for 5 minutes; and cooled to 4° C. The resulting PCR product was purified using a QIAquick Multiwell PCR Purification kit (Qiagen) according to the manufacturer's protocol and was used in subsequent steps. The amplified PCR product was cleaved with BamHI and XhoI restriction enzymes and inserted into a pET21b (Novagen) vector that was also cleaved with BamHI and XhoI, to construct the vector, pET12b-cherry.

PCR amplification was also performed using as template the plasmid pCSZif268 (Kim and Pabo, 1998, PNAS, 95:2812-2817), a ZIF268F primer (SEQ ID NO. 3) including a nucleotide sequence cleavable with NdeI and a ZIF268R primer (SEQ ID NO. 4) coding for part of the (Gly₂Ser)₅ linker and including a nucleotide sequence cleavable with BamHI, to obtain the target nucleotide sequence-binding protein. The amplification was performed using a GENEAMP® PCR System 9700 (Applied Biosystems) under the following PCR conditions: at 95° C. for 5 minutes; at 95° C. for 20 seconds; repeated 30 times at 68° C. for 2 minutes; at 68° C. for 5 minutes; and cooled to 4° C. The resulting PCR product was purified using a QIAquick Multiwell PCR Purification kit (Qiagen) according to the manufacturer's protocol and was used in subsequent steps. The amplified PCR product was cleaved with BamHI and Nde restriction enzymes and inserted into the pET12b-cherry vector, cleaved with the same restriction enzymes to construct the vector, pET12b-Zif268-cherry.

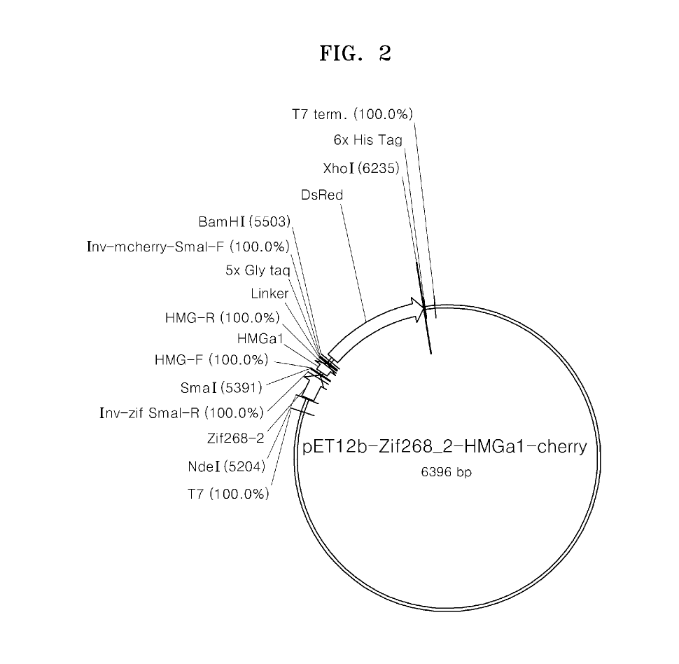

Amplification was also performed using cDNA, constructed using total RNA from blood and a reverse transcriptase (Invitrogen), as the template, an HMGF primer (SEQ ID NO. 3) including a nucleotide sequence coding for a Gly₅ linker and an HMGR primer (SEQ ID NO. 6) to obtain an HMGa1 domain amplicon. The amplification was performed using a GENEAMP® PCR System 9700 (Applied Biosystems) under the following PCR conditions: at 95° C. for 15 minutes; at 95° C. for 20 seconds; at 58° C. for 30 seconds; repeated 35 times at 72° C. for 30 seconds; at 72° C. for 3 minutes; and cooled to 4° C. The resulting PCR product was purified using a QIAquick Multiwell PCR Purification kit (Qiagen) according to the manufacturer's protocol and was used in subsequent steps. The amplified PCR product was cleaved with SmaI restriction enzyme and inserted into the pET12b-Zif268-cherry vector that was also cleaved with SmaI to construct the vector, pET12b-Zif268-HMGa1-cherry (see FIG. 2).

An expression vector for a fusion protein including eHU protein, instead of the HMGa1 domain, was constructed in the same manner, except that, instead of the polynucleotide fragment coding for the HMGa1 domain, a polynucleotide fragment coding for the eHU protein was inserted into the pET12b-Zif268-cherry vector. The constructed expression vector was named pET12b-Zif268-eHU-cherry. Primers (SEQ ID NOs. 7 and 8) and the template cDNA disclosed above were used to amplify the polynucleotide fragment coding for the eHU protein.

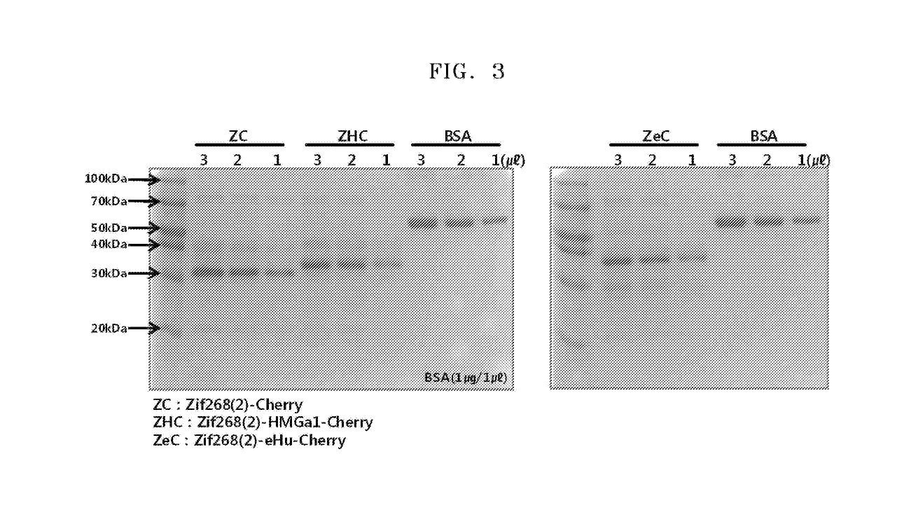

In order to use either pET12b-Zif268-HMGa1-cherry or pET12b-Zif268-eHU-cherry to over-express the encoded fusion proteins, these vectors were transformed into E. coli BL21 (DE3). Luria Broth (LB) liquid medium to which 50 μg/ml of ampicillin was added was used as the culture medium. When the optical density (O.D., absorbance) reached a value of 0.5 at a 600-nm wavelength, 0.5 mM isopropyl-β-d-thiogalactopyranoside (IPTG) was added to the culture medium. The transformed E. coli BL21 (DE3) culture was further grown at about 25° C. for about 16 hours. After sonication in a 25 mM Tris-HCl buffer solution (pH 8.0), the cell culture was centrifuged (at 10,000×g) to obtain a supernatant. The supernatant was loaded on a Ni²⁺-NTA superflow column (Qiagen) equilibrated with the 25 mM Tris-HCl buffer solution, and was then washed with a wash buffer solution in a volume five times higher than that of the column. Then, an elution buffer solution (25 mM Tris-HCl (pH 8.0), 2.5 mM β-mercaptoethanol, 125 mM imidazole, and 150 mM NaCl) was loaded to elute bound protein. Protein fractions were collected and filtered using AMICON® Ultra-15 Centrifugal Filters (Milipore) to remove salts. Then, the desalted fractions were concentrated. The concentrated fusion protein was dissolved and stored in storage solution A (25 mM Tris-HCl (pH 8.0), 2.5 mM β-mercaptoethanol, 125 mM imidazole, 150 mM NaCl, and 50% glycerol) or storage solution B (20 mM Tris-HCl (pH 7.5), 1 mM DTT, 100 mM NaCl, and 50% glycerol). The concentration of the purified protein was quantified using bovine serum albumin (BSA) as the standard protein. FIG. 3 shows that the fusion proteins disclosed herein had a molecular weight of about 35 kDa and were separated with a high purity.

### Example 2

### Measurement of the Binding Affinity of the Fusion Proteins to Target Nucleic Acid

Affinity of the fusion proteins prepared in Example 1 to a target nucleic acid nucleotide sequence was measured using a surface plasmon resonance (SPR) system (Biacore) to determine the equilibrium dissociation constant, K_{d}, for forming the fusion protein-target nucleic acid complex. After immobilization of the target nucleic acid (SEQ ID NO. 9) to a CM5 chip, the fusion proteins were loaded into the microfluidic chamber of the SPR. The K_{d} values of the fusion proteins including the HMGa1 domain or the eHU protein, respectively, were calculated from the results based on a protocol of the manufacturer, and were found to be about 140 nM and 860 nM, respectively. Therefore, these experimental results indicate that the fusion proteins of Example 1 have strong affinity to the target nucleic acid.

As described above, by using a kit and a method of determining a nucleotide sequence of a target nucleic acid, according to one or more of the above embodiments of the present disclosure, a nucleotide sequence of a target nucleic acid in a sample may be more efficiently determined.

The terminology used herein is for the purpose of describing particular embodiments only and is not intended to be limiting of the invention. The terms “a” and “an” do not denote a limitation of quantity, but rather denote the presence of at least one of the referenced item. The terms “comprising”, “having”, “including”, and “containing” are to be construed as open-ended terms (i.e. meaning “including, but not limited to”).

Recitation of ranges of values are merely intended to serve as a shorthand method of referring individually to each separate value falling within the range, unless otherwise indicated herein, and each separate value is incorporated into the specification as if it were individually recited herein. The endpoints of all ranges are included within the range and independently combinable.

All methods described herein can be performed in a suitable order unless otherwise indicated herein or otherwise clearly contradicted by context. No language in the specification should be construed as indicating any non-claimed element as essential to the practice of the invention as used herein.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

It should be understood that the exemplary embodiments described herein should be considered in a descriptive sense only and not for purposes of limitation. Descriptions of features or aspects within each embodiment should typically be considered as available for other similar features or aspects in other embodiments.

What is claimed is:

1. A kit for determining a nucleotide sequence of a target nucleic acid, the kit comprising:
at least two nucleic acid-binding probes, wherein each nucleic acid-binding probe comprises
a first protein that specifically binds a nucleotide sequence consisting of n bases, wherein n is an integer ranging from 3 to 12;
a second protein, linked to a terminus of the first protein, that non-specifically binds to a minor groove of nucleic acid; and
a detectable tag linked to a terminus of the second protein.

2. The kit of claim 1, wherein n is 6.

3. The kit of claim 1, wherein the first protein may include at least one nucleic acid-binding motif selected from the group consisting of a zinc finger motif, a helix-turn-helix motif, a helix-loop-helix motif, a leucine zipper motif, a nucleic acid-binding motif of a restriction endonuclease, a TATA-binding protein (TBP) domain, and combinations thereof.

4. The kit of claim 1, wherein the first protein comprises two zinc finger motifs.

5. The kit of claim 1, wherein the detectable tag comprises at least one selected from the group consisting of a colored bead, a chromophore, a fluorescent material, a phosphorescent material, an electrically detectable molecule, a molecule providing modified fluorescence-polarization or modified light-diffusion, and a quantum dot.

6. The kit of claim 1, wherein the detectable tag comprises at least one selected from the group consisting of Alexa Fluor 350, Alexa Fluor 430, Alexa Fluor 488, Alexa Fluor 532, Alexa Fluor 546, Alexa Fluor 568, Alexa Fluor 594, Alexa Fluor 633, Alexa Fluor 647, Alexa Fluor 660, Alexa Fluor 680, Cy2, Cy3.18, Cy3.5, Cy3, Cy5.18, Cy5.5, Cy5, Cy7, mCherry, Oregon Green, Oregon Green 488-X, Oregon Green, Oregon Green 488, Oregon Green 500, Oregon Green 514, SYTO 11, SYTO 12, SYTO 13, SYTO 14, SYTO 15, SYTO 16, SYTO 17, SYTO 18, SYTO 20, SYTO 21, SYTO 22, SYTO 23, SYTO 24, SYTO 25, SYTO 40, SYTO 41, SYTO 42, SYTO 43, SYTO 44, SYTO 45, SYTO 59, SYTO 60, SYTO 61, SYTO 62, SYTO 63, SYTO 64, SYTO 80, SYTO 81, SYTO 82, SYTO 83, SYTO 84, SYTO 85, SYTOX Blue, SYTOX Green, SYTOX Orange, SYBR Green YO-PRO-1, YO-PRO-3, YOYO-1, YOYO-3, and thiazole orange.

7. The kit of claim 1, wherein the detectable tag is linked to the terminus of the second protein via a linker.

8. The kit of claim 1, wherein the second protein comprises at least one selected from the group consisting of a homeobox protein, a high mobility group (HMG) protein, a HU protein, a histone-fold protein, a polymerase cleft protein, and a protein including sequence non-specific zincfinger xfin31, β-barrel CspA, an arginine-rich region, and a RGG motif.

9. A method of determining a nucleotide sequence of a target nucleic acid, the method comprising:
contacting a target nucleic acid, whose nucleotide sequence is to be detected, with the at least two nucleic acid-binding probes of the kit of claim 1;
detecting a signal from the detectable tag linked to each of the at least two nucleic acid-binding probes; and
converting the detected signal into a nucleotide sequence present in the target nucleic acid.

10. The method of claim 9, wherein the target nucleic acid comprises a double-stranded polynucleotide.

11. The method of claim 9, wherein the target nucleic acid has a length of about 100 bp to about 10 Mb.

12. The method of claim 9, wherein the target nucleic acid comprises a second detectable tag.

13. The method of claim 12, wherein the second detectable tag comprises at least one selected from the group consisting of a colored bead, a chromophore, a fluorescent material, a phosphorescent material, an electrically detectable molecule, a molecule providing modified fluorescence-polarization or modified light-diffusion, and a quantum dot.

14. The method of claim 9, wherein the signal is generated by fluorescent resonance energy transfer (FRET) between the detectable tag bound to each of the at least two nucleic acid-binding probes and the second detectable tag bound to the target nucleic acid.

15. The method of claim 9, further comprising outputting the nucleotide sequence to a user.

16. A method of determining the presence or absence of a nucleotide sequence in a target nucleic acid, the method comprising:
contacting a target nucleic acid with the at least two nucleic acid-binding probes of the kit of claim 1;
detecting a signal from the detectable tag linked to one of the nucleic acid-binding probes; and
determining that the nucleotide sequence specifically bound by the nucleic acid-binding probe is present in the target nucleic acid when the signal from the detectable tag linked to the nucleic acid-binding probe indicates binding of the nucleic acid-binding probe to the target nucleic acid, or
determining that the nucleotide sequence specifically bound by one of the nucleic acid-binding probes is absent from the target nucleic acid when the signal from the detectable tag linked to the nucleic acid-binding probe does not indicate binding of the nucleic acid-binding probe to the target nucleic acid.
